(19) United States
(12) Patent Application Publication
(10) Pub. No.: US 2011/0281743 A1
(43) Pub. Date: Nov. 17, 2011

(54) SYSTEMS AND METHODS FOR ISOLATING CELLS IN CELL COLONIES IN CULTURE

(76) Inventors: Nancy Allbritton, Chapel Hill, NC (US); Christopher Sims, Chapel Hill, NC (US); Wei Xu, Chapel Hill, NC (US); Hamed Shadpour, Tustin, CA (US)

(21) Appl. No.: 13/130,345

(22) PCT Filed: Dec. 10, 2009
(86) PCT No.: PCT/US09/67482

§ 371 Date: Jul. 21, 2011

Related U.S. Application Data
(60) Provisional application No. 61/121,395, filed on Dec. 10, 2008.

(51) Int. Cl. C40B 30/00; C12Q 1/68; H01J 49/26; C40B 60/12; H01J 37/26; C12Q 1/02; G01N 33/53

(52) U.S. Cl. 506/7; 435/29; 435/6.12; 435/6.1; 435/7.1; 506/39; 250/307; 250/282

(57) ABSTRACT

Selecting and propagating a cell colony of interest from among a plurality of cell colonies carried on a common substrate in culture is carried out by: (a) selecting a cell colony of interest from among the plurality of cell colonies; (b) isolating a cell subset from the cell colony of interest; (c) analyzing (for example, by a destructive analysis) the cell subset isolated from the cell colony of interest to confirm the presence or absence of a desired feature therein; and then (d) propagating the cell colony of interest when the desired feature is present in the cell subset. A micropallet apparatus may include: (a) a substrate; (b) a plurality of discrete arrays formed on the substrate, each of the arrays comprising a plurality of releasable pallets, and (c) a plurality of gap forming regions, wherein the gap forming regions surround the pallets and separate the pallets from one another.

### BACKGROUND OF THE INVENTION

Manual methods of cell sampling such as cloning rings and colony picking require colonies of several thousand cells. The entire colony must be collected and trypsinized to separate the cells. Only then can the cells be split to gather a sample for analysis. The remaining cells must be placed back in culture or frozen pending the results of the analysis.

Laser capture microdissection (LCM) (Arcturus; Mountain View, Calif.) has enabled single cells or small groups of selected cells to be obtained from tissue sections for genetic and proteomic studies, although most applications utilize fixed or frozen specimens. Protocols for use with live cells have been published, but are very low throughput and not suitable for isolating large numbers of single, living cells.

P.A.L.M. Microlaser Technologies (Bernried, Germany) markets an instrument that uses a laser to cut out a region of interest from a tissue section and then generate a shock wave that “catapults” the cells into an overlying collection device. Again most of the work with this technique has utilized fixed specimens, but collection of living cells has been demonstrated. Cells are subjected to stress due to the direct effects of the shock wave and desiccation from removal of fluid overlying the sample during collection.

Cyntellect, Inc. (San Diego, Calif.) uses negative selection to enrich samples for cells of interest, and has shown enrichment of populations of hybridoma cells for antibody production. This technique suffers from the almost impossible feat of ablating all unwanted cells making contamination problematic.

ClonePix (Genetix, Hampshire, UK) is an instrument originally developed as a high-throughput tool for aspirating bacterial and yeast colonies from agar plates, but it is now being marketed for isolation of mammalian cells. This automated system uses image recognition to guide a suction pipet that aspirates colonies of loosely adherent cells from plates with or without the addition of a “proprietary release buffer”. The system has only been demonstrated with mammalian cells that grow in loosely adherent clusters or suspension-adapted versions of adherent cells growing in a semi-solid methylcellulose media. It is not applicable to the vast majority of mammalian cells.

In view of the foregoing, there is needed a way to select live, adherent cells singly or in small groups from a larger colony based on morphology, surface markers, and dynamic characteristics followed by collection with minimal perturbation for expansion or further analysis.

### SUMMARY OF THE INVENTION

A first aspect of the invention is a method for selecting and propagating a cell colony of interest from among a plurality of cell colonies carried on a common substrate in culture, the method comprising the steps of: (a) selecting a cell colony of interest from among the plurality of cell colonies; (b) isolating a cell subset from the cell colony of interest; (c) analyzing (for example, by a destructive analysis) the cell subset isolated from the cell colony of interest to confirm the presence or absence of a desired feature therein; and then (d) propagating the cell colony of interest when the desired feature is present in the cell subset.

A further aspect of the invention is a micropallet apparatus, which may be used to carry out a method as described above. The apparatus comprises: (a) a substrate; (b) a plurality of discrete arrays formed on the substrate, each of the arrays comprising a plurality of releasable pallets, and (c) a plurality of gap forming regions, wherein the gap forming regions surround the pallets and separate the pallets from one another.

The present invention is explained in greater detail in the drawings herein and the specification set forth below.

### BRIEF DESCRIPTION OF THE DRAWINGS

FIG. 1. Sample plate for selecting cells from a colony. A) Cell colonies (orange color regions) grow on the segregated micropallet arrays B) One or more micropallets containing a portion of a colony is(are) released. The micropallet and cell(s) are collected for analysis to identify target colonies. C) Once the targeted cell colonies are identified, micropallets with the main body of the colony are released and collected for further use such as genetic analysis.

FIG. 2. An example design of a plate composed of segregated micropallet arrays. A) Shown is a top view of the plate. The purple region represents walls that segregate the individual arrays while the yellow region represents the walls between the micropallets within each array. Micropallets are represented in blue. B), C) and D) are three example designs of individual micropallet arrays on the plate. In B & C, micropallets are composed of a single layer of material forming the individual pallets. D) & E) show top and side views, respectively, of an array containing micropallets composed of multiple (i.e., 2) layers.

FIG. 3. Examples of wall dimensions on a segregated array plate. The width of the walls between arrays can be smaller A) or larger B) than the size of each single segregated array. The height of the walls could be either the same (A,B) or higher C) than the micropallets. The materials of the walls could be made of single material (A, B&C) or the combination of two or more different materials D) Red region and purple region represent two different wall materials.

FIG. 4. Microscopic photographs of different designs of segregated micropallet arrays made from 1002F photoresist. The sizes of the micropallets inside each single segregated array can be varied as shown (A-E). The surface of the micropallets can be flat or possess micropatterns to modify surface roughness (F-I). The walls segregating the arrays can be formed by a solid material such as the 1002F photoresist (K) or the combination of gas and solid, i.e., 1002F photoresist (J). The height of the walls can be greater than the micropallets (K).

FIG. 5. Colony overgrowth and sampling. A) Shown is a micrograph of a murine embryonic stem (ES) cell colony which has overgrown its original pallet and spread to several bordering pallets. B) Shown is the same field of view immediately after a single border pallet (lower, left) was released using the pulsed laser.

FIG. 6A-6L show various alternate embodiments of the present invention.

FIG. 7A-7D. (a) Transmitted and fluorescent light images of cell colony growing on the segregated micropallet unit; (b) after release of the “cutting” micropallet; (c) at 72 hr, the portion of the cell colony remaining on the array continued to grew; and (d) at 72 hr the released portion of the cell colony continued to grow from the released and collected segregated micropallet.

### DETAILED DESCRIPTION OF THE INVENTION

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

Like numbers refer to like elements throughout. In the figures, the thickness of certain lines, layers, components, elements or features may be exaggerated for clarity. Where used, broken lines illustrate optional features or operations unless specified otherwise.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

Spatially relative terms, such as “under,” “below,” “lower,” “over,” “upper” and the like, may be used herein for ease of description to describe an element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus the exemplary term “under” can encompass both an orientation of over and under. The device may otherwise be oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only, unless specifically indicated otherwise.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section. Thus, a first element, component, region, layer or section discussed herein could be termed a second element, component, region, layer or section without departing from the teachings of the present invention. The sequence of operations (or steps) is not limited to the order presented in the claims or figures unless specifically indicated otherwise.

“Carried on” as used herein to describes cells on a substrate includes cells adhered directly to the substrate, or cells adhered to the substrate through one or more intervening layers or carriers (which carrier may in turn be releasably adhered to the substrate). Examples of such carriers include, but are not limited to liquid particles, solid particles, cleavable molecules and combinations thereof (including multi-layer carriers).

“Destructive analysis” as used herein refers to a technique for studying or analyzing cells in which the cells are killed, cannot subsequently be propagated, or are so altered as to not be useful for further propagation or investigation. Examples of destructive analysis include but are not limited to polymerase chain reaction (PCR), intracellular immunostaining, mass spectrometry, mRNA expression, electron microscopy, electrophoretic analysis, and DNA analysis.

“Desired feature” as used herein may be a predetermined genotype and/or predetermined phenotype (e.g., as caused by mutagenesis with a chemical or physical mutagen such as radiation), a predetermined or preselected point mutation or single nucleotide polymorphism (SNP), the presence of a heterologous nucleic acid, the stable integration of a heterologous nucleic acid, the transient or stable expression of a heterologous nucleic acid, etc.

“Heterologous nucleic acid” as used herein refers to an exogeneous nucleic acid (DNA, RNA) inserted into a cell by application of a laboratory procedure such as microparticle bombardment, electroporation, etc. Such a heterologous nucleic acid is typically a recombinant nucleic acid carried by a vector, such as a plasmid, virus, retrovirus, etc.

As noted above, a first aspect of the invention is a method for selecting and propagating a cell colony of interest from among a plurality of cell colonies carried on a common substrate in culture, the method comprising the steps of:

(a) selecting a cell colony of interest from among the plurality of cell colonies;

(b) isolating a cell subset from the cell colony of interest;

(c) analyzing (for example, by a destructive analysis) the cell subset isolated from the cell colony of interest to confirm the presence or absence of a desired feature therein; and then

(d) propagating the cell colony of interest when the desired feature is present in the cell subset.

In some embodiments of the foregoing, the plurality of cell colonies comprises not more than 5, 10 or 20 colonies; In other embodiments of the foregoing, the plurality of cell colonies comprises at least 50 or 100 cell colonies. In some embodiments of the foregoing, the cell colony of interest comprises not more than 10, 100, 1000, or 10,000 cells.

In some embodiments of the foregoing, the step of isolating a cell subset is carried out by releasing a portion of cells from the colony of interest, while maintaining the remainder of the colony of interest on the substrate. In some embodiments, each of the plurality of colonies are grown on a plurality of carriers (one example of which is a pallet) releasably connected to the common substrate; and the releasing step is carried out by releasing the carrier from the substrate to thereby release a portion of cells from the colony of interest. In other embodiments, the step of releasing a portion is carried out by laser cutting of the colony.

A particular aspect of the present invention is a method for isolating a cell subset from a live cell colony grown in culture, comprising the steps of: (a) providing a micropallet apparatus, the apparatus comprising a: (i) a substrate; (ii) a plurality of discrete arrays formed on the substrate, each of the arrays comprising a plurality of pallets releasably connected to the substrate; and (iii) a cell colony of interest adhered to one of the arrays, the cell colony of interest spanning at least two pallets; (b) selecting a subset of at least one pallet from the at least two pallets to which the cell colony of interest is adhered; (c) releasing the selected subset of pallets; then (d) collecting at least one cell from the selected subset of pallets to thereby isolate a cell subset from the colony. In some embodiments, then optionally (e) analyzing (for example, by a destructive analysis) the cell subset isolated from the cell colony of interest to confirm the presence or absence of a desired feature therein, and then optionally (f) propagating the cell colony of interest when the desired feature is present in the selected cell subset.

In some embodiments of the foregoing, pallets of each of the arrays are separated from one another by gaps. In some embodiments of the foregoing, the arrays are separated from one another by walls. In some embodiments of the foregoing, the pallet is connected to the substrate at a release point, and the releasing step is carried out by directing a high energy laser at the release point.

A further aspect of the invention is a micropallet apparatus, comprising: (a) a substrate; (b) a plurality of discrete arrays formed on the substrate, each of the arrays comprising a plurality of releasable pallets, (c) a plurality of gap forming regions, wherein the gap forming regions surround the pallets and separate the pallets from one another, and optionally (d) a plurality of walls, wherein the walls surround the arrays and separate the arrays from one another.

In some embodiments of the foregoing, the pallets are transparent (e.g., optically transparent so that a laser may pass therethrough; and/or visually transparent so that the pallets may be seen through by a human observer aided by light microscopy).

In some embodiments, the pallets are formed from a photoresist resin, a photoactive compound, and a solvent.

In some embodiments, the pallets are formed from EPON resin 1002F, photoinitiator triarylsulfonium hexafluoroantimonate, and γ-butyrolactone.

In some embodiments, the pallets have heights in the range of 1 to 400 micrometers.

The surface of the pallets may be modified to enhance cell culture, e.g., by texturing and/or coating the top surface thereof.

In some embodiments, the gap forming regions are configured to allow cells to spread over multiple pallets.

In some embodiments, the gap forming regions are formed from a gas, a liquid, a hydrogel, a solid material or combination thereof.

In some embodiments, the walls are configured to prevent cell colonies from spreading onto adjacent arrays. In some embodiments the walls are formed from a gas, a hydrogel, a solid material, or combination thereof. Optionally, the walls may comprise a cell adhesion resistant material, such as a PEG hydrogel.

In some embodiments the apparatus may further comprise a collection plate connected to the micropallet apparatus e.g., by means of a clamp, cooperating interlocking connecting portions such as threads formed on each, combinations thereof, etc. The collection plate may, in turn, comprise a plurality of wells, with the plurality of wells are aligned with the plurality of arrays.

Particular embodiments of the invention provide a method for isolating cells from a colony of cells in culture. In some embodiments this sampling procedure can be performed even when the colony is too small to be manipulated by traditional means such as cloning rings or pipette picking. Furthermore, the colony can be sampled while maintaining the viability of the parent colony. An advantage of this method is that it broadens the methods by which a small colony of interest can be identified through the use of analysis methods that would normally be destructive to the cells. In this regard, the viability of the parent colony is maintained even if the analysis of the sampled cells is destructive. By collecting a sample of the cells, the colony can be analyzed by a variety of techniques including, but not limited to, PCR, intracellular immunostaining, mass spectrometry, mRNA expression, electron microscopy, electrophoretic methods, DNA analysis, and others. Nevertheless, the sampling procedure can maintain viability of the sampled cells if desired for nondestructive analyses or sub-culturing.

In some embodiments, by virtue of the microengineered scale of this method, sampling can be performed much earlier in the life of the colony, which can reduce reagent and manpower costs in culturing the cells. This capacity also provides the ability to analyze the colony much earlier than conventional approaches. Furthermore, the invention enables a large number of colonies to be efficiently sampled so that colonies of interest can be segregated from colonies of no further value after only a short period in culture. An example of the field of use for the invention is the early detection and selection of clonal colonies of cells stably expressing a transfected gene.

The current invention differs from prior micropallet methods and apparatus in that an element of the prior systems was that an individual cell or colony of cells must be localized to a single pallet. Cells or colonies spanning two or more pallets were deemed failures. Central to embodiments of the present invention is that a colony of cells must span two or more pallets. In the prior systems the cell or colony was localized to single pallets by virtue of an intervening region between the pallets that prevented migration of cells from one pallet to another. In this manner, individual micropallets when released carried an entire cell or entire colony that was then collected for clonal expansion. In the current application, modifications to the micropallet array enable the colony of cells to grow over multiple pallets. To sample a portion of the colony, one or more pallets on which the colony has spread is/are released. The released pallet carries with it some of the cells from that colony which are then collected. In this manner, a portion of the cells making up the colony are sampled to be used for biological analysis of that colony or for sub-culturing the colony.

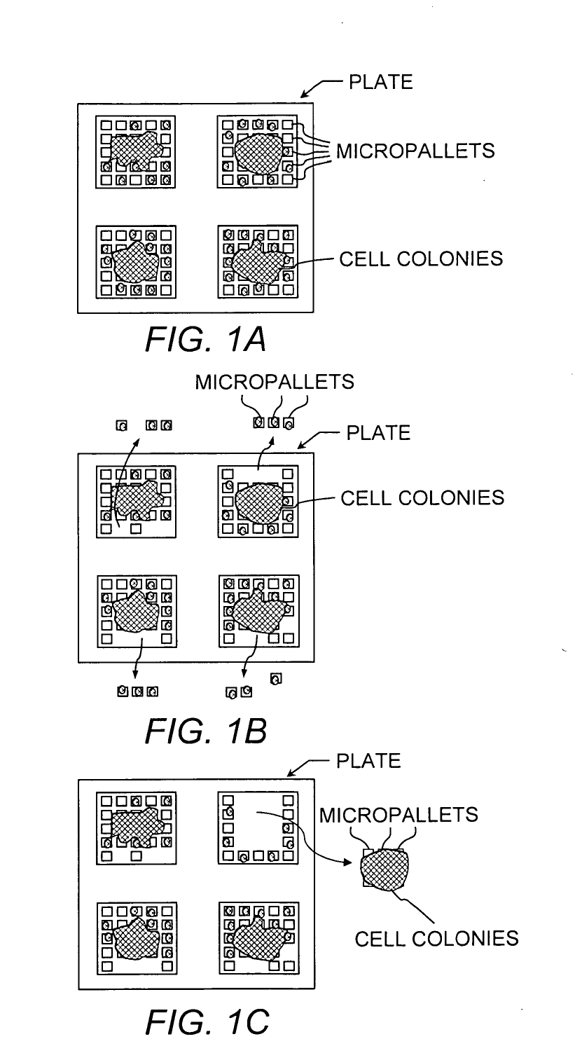

One application for this invention is for the isolation of homologously recombined stem cells. The cells can be genetically modified to carry a heterologous nucleic acid and then cultured as clonal colonies on the micropallet array where they overgrow multiple pallets (FIG. 1A). A sample composed of a portion of a colony of stem cells is released by using a laser to dislodge the underlying border pallet (FIG. 1B). Collected cells are then analyzed by genetic techniques for the presence of homologous recombination. Colonies composed of homologously recombined cells are collected (FIG. 1C) and used to create genetically modified ES cell lines or implanted into embryos for the purpose of creating genetically engineered mouse models.

A variety of features can be incorporated into the pallet array to improve cell culture and colony sampling as follows. The design of the micropallet array is further specified as a patterned plate composed of segregated arrays, each array surrounded by walls. In this design, segregated arrays of micropallets are created on a plate and walls are created between these segregated arrays to isolate them. Inside each segregated array, micropallets may also be surrounded by walls. Walls between or inside segregated arrays could be of the same or different materials from the micropallets. The purpose of the segregated design is to optimize the culture and collection of cells and colonies on the patterned plate.

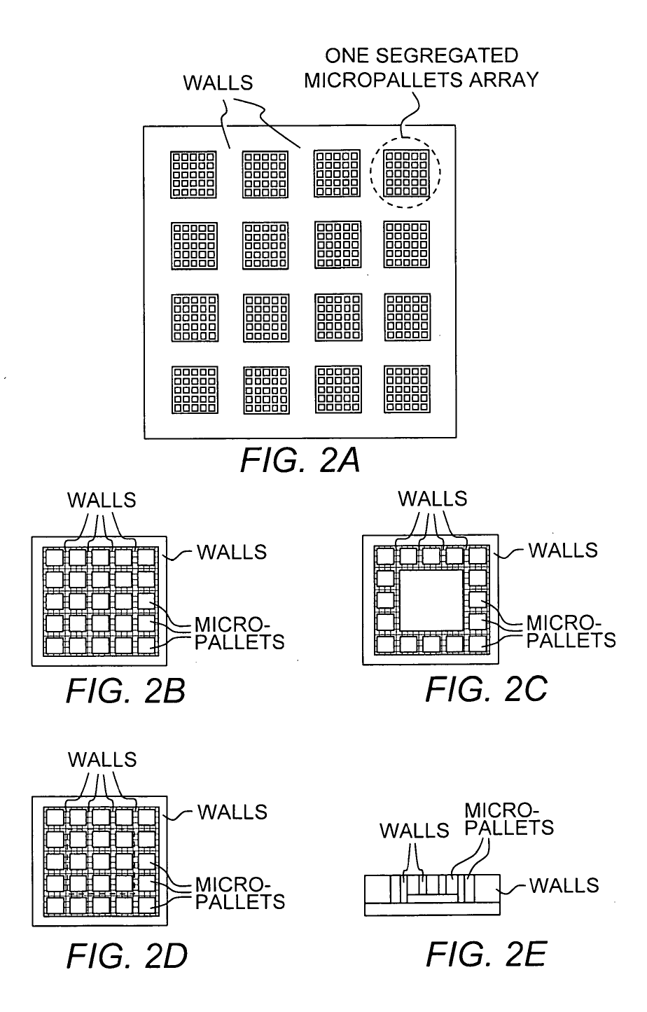

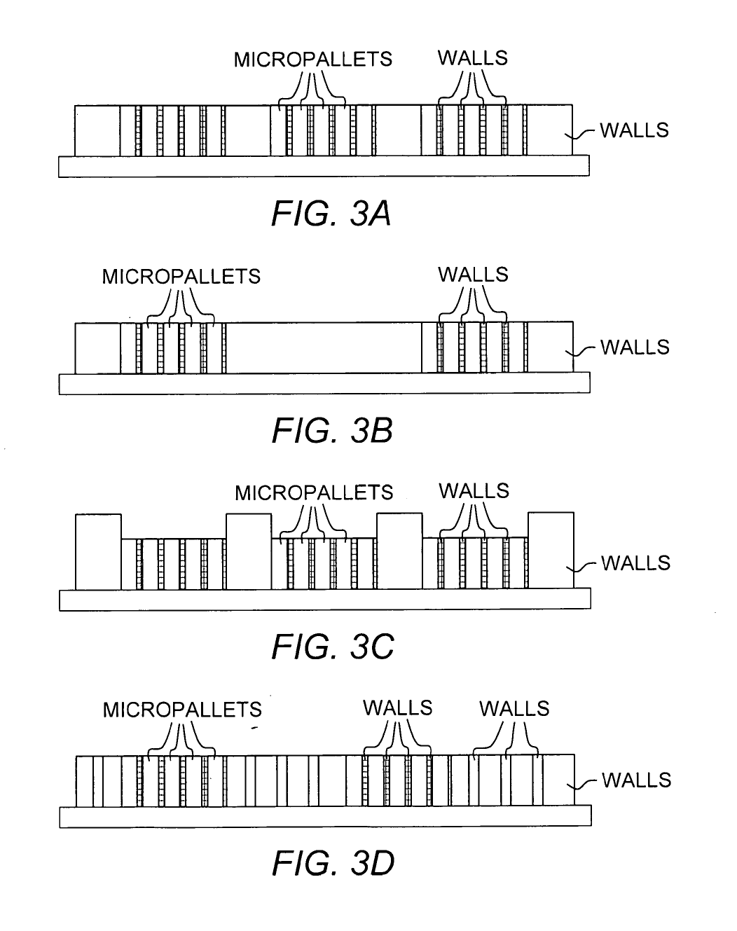

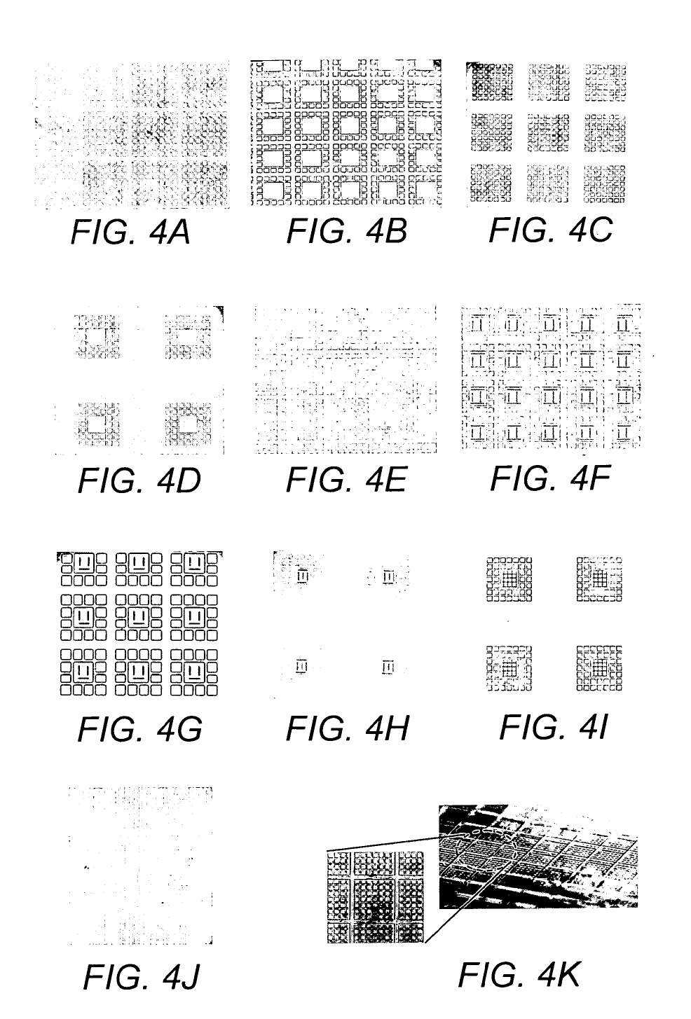

The overview of the design of an embodiment of a segregated micropallet array plate is shown in FIGS. 2 and 3. Arrays composed of micropallets are created on a plate. The individual arrays on the plate may be the same dimension or of different dimensions. Within each segregated array of micropallets, the size, the shape, the surface roughness, the surface pattern, the number of micropallets and the layers of the micropallet can be similar or can vary. Three potential designs of a segregated micropallet array are shown as examples in FIGS. 2 B, C & D and FIG. 3. The walls between the arrays and between micropallets could be made by a variety of materials such as, but not limited to, a gas (e.g., air), a hydrogel (e.g. poly[ethylene glycol] or PEG), a polymer (e.g. the photoresists 1002F or SU-8), a liquid (e.g., immersion oil), or a combination of these materials. The height of the walls between the arrays can be made the same, lower or higher than the height of the micropallets. The spacing between the micropallets within individual arrays and between the arrays themselves can be varied to meet the unique requirements of different applications. FIG. 4 shows photomicrographs of segregated micropallet arrays created from 1002F photoresist.

This invention describes a novel method to isolate cells from cell colonies in culture when the colonies are too small to manipulate by other means. Furthermore, the colony can be sampled while maintaining the viability of both the remaining colony and the isolated cells if so desired. With this approach, small colonies can be sampled and analyzed for a characteristic using a destructive analysis technique such as PCR, immunohistochemical staining, and others while maintaining the parent colony in culture. In this manner, colonies can be evaluated at very early times for a characteristic of interest, such as presence of a transfected gene, to reduce manpower and reagent costs associated with maintaining the cells in culture.

This invention can be applied in almost every field where cell culture and cloning is used. Such fields include, but are not limited to, basic cell biology, stem cell research, cancer research, cell-based assays, drug development, drug screening, genetic medicine and regenerative medicine.

Some cell types may be more adherent to each other than to the bordering pallets. This may result in the cell mass remaining adherent interfering with release and collection of a portion of the colony. This issue can be addressed by use of surface coatings or roughening to increase cell adhesion to the micropallets.

### Experimental

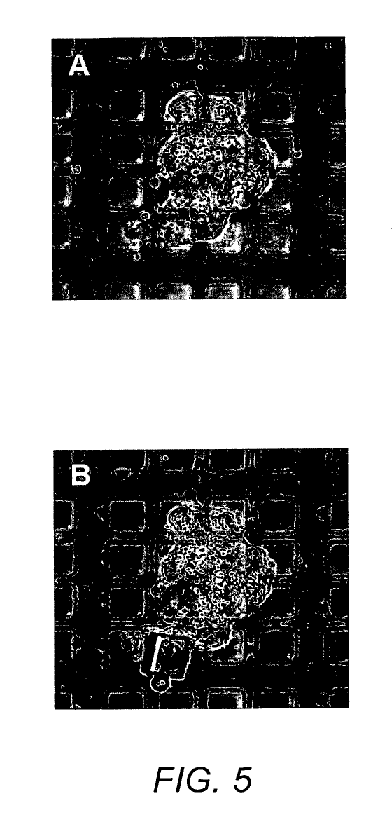

In one example of the present invention, murine ES cells were cultured on micropallet arrays. The design of the pallet array was modified to provide smaller gaps between the pallets so that the intervening virtual air walls did not present an effective barrier for colony spread. This design had central pallets of 100 μm length and 75 μm height on which ES cell colonies were cultured. Each of these larger central pallets was surrounded by 12 small (40 μm length, 75 μm height, 20 μm gap) bordering pallets which would be released to obtain a portion of the colony. After 3 days of culture, the colonies frequently exceeded the pallet growth area of the central pallet and the cells of an individual colony spread onto the smaller bordering pallets (FIG. 5A). After 6 days, a bordering pallet containing a portion of a colony was released. Border pallets released in this manner carried with them their mass of attached cells leaving the remaining cells of the colony behind (FIG. 5B). This data demonstrates colonies can be cultured over multiple pallets and that sampling a portion of the colony during culture is feasible.

As described above, one embodiment of the invention was demonstrated by the application of a micropallet array to isolate cells from a colony of murine ES cells. While murine ES cells were used as a model cell type, other cell types including human stem cells and primary cells (e.g., adult and embryonic stem cells, cells taken directly from patients, or from animal models) tissue culture cell lines, plant cells, yeast cells, and other cell types could be used. Various cell types can be obtained from human or animal sources such as, but not limited to embryo or tissue sources, or they can be originated from plant tissues. Cells are cultured on the segregated micropallet array plate by adding a suspension of cells and allowing the cells to settle and attach to the micropallets. By controlling the density of the cell suspension, single or multiple cells can be positioned on the micropallets. It is also possible to deposit cells by pipette, fluid flow, dielectrophoresis, magnetic manipulation, or other cell manipulation techniques. The arrays in this application are designed so that the width of the gaps between micropallets is narrow enough to enable the stem cell colony to spread over the micropallets as the colony enlarges. In this situation, the region forming the gap may be filled in with gas, liquid, or a solid material that is distinct from the micropallets. In contrast, the walls segregating the individual arrays are created to be of sufficient dimension to prevent the extension of the colony to other arrays. The height of these walls may also be of greater magnitude to aid in segregating the colonies present on different arrays. Furthermore, the material composing this segregating wall is chosen for its property of resisting cell adhesion, for example a PEG hydrogel. As a result, any given cell colony is confined to grow only over a single micropallet array. Once the colonies grow to sufficient size, one or more micropallets can be released from the plate carrying with it(them) the attached cell(s) for further culture or analysis (e.g., PCR, Southern blot, or other analysis technique). The analyses may cover a broad range of genomics, proteomics, biochemical or other study and can be performed using any of a variety of existing or future technologies. The purpose of the analysis step is to determine a genetic, proteomic, biochemical, or other characteristic that serves to identify the colony as composed of cells of interest. Once the targeted colonies are identified, a portion or all cells from the colony can be collected by means of the release of micropallets or other removal technique. The collected cells are then expanded or used for the desired application. In the current example, murine ES cells were cultured and grown into colonies.

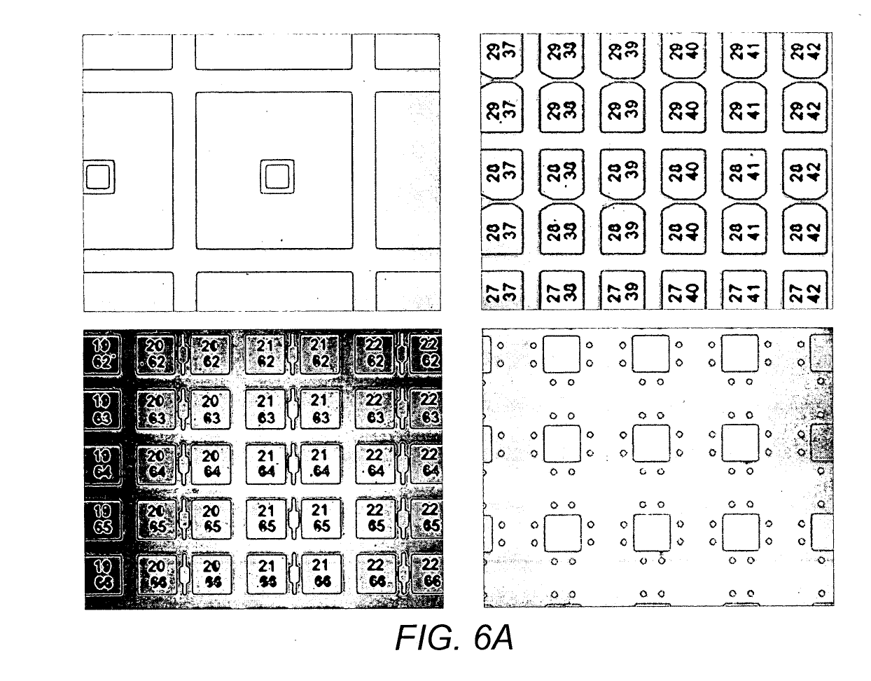

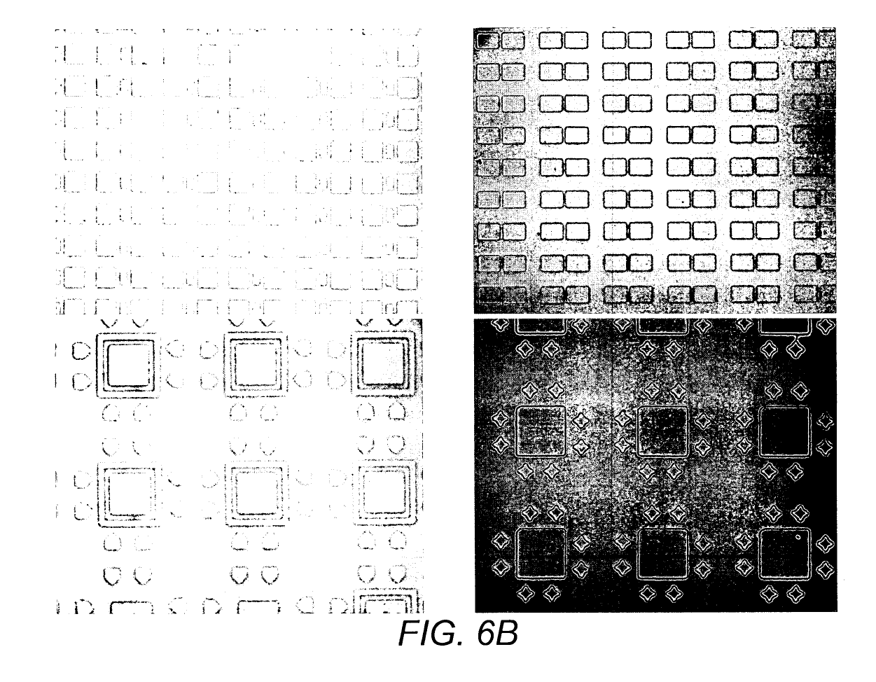

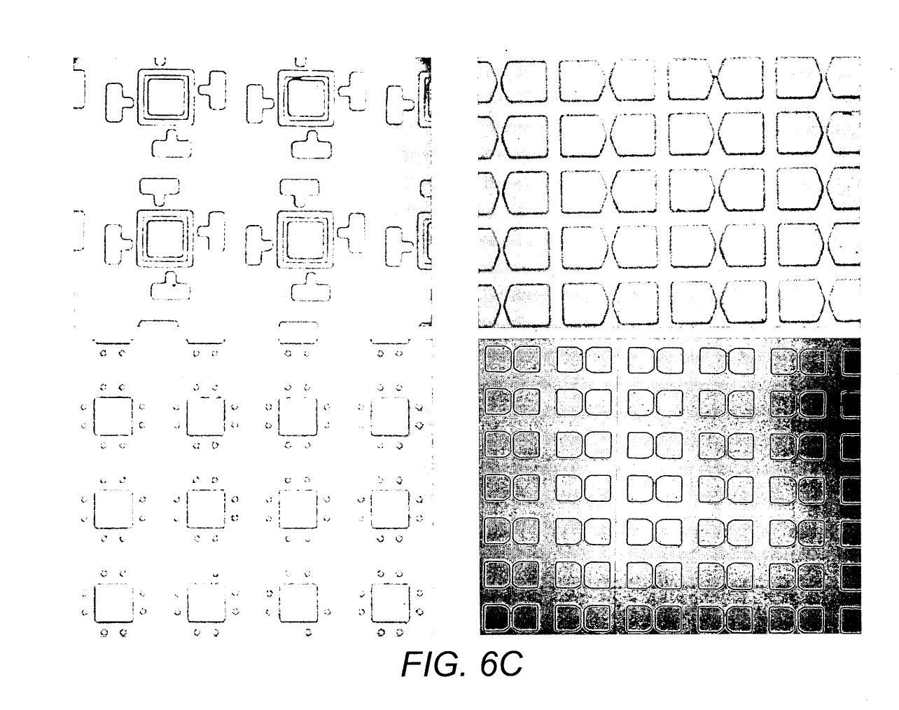

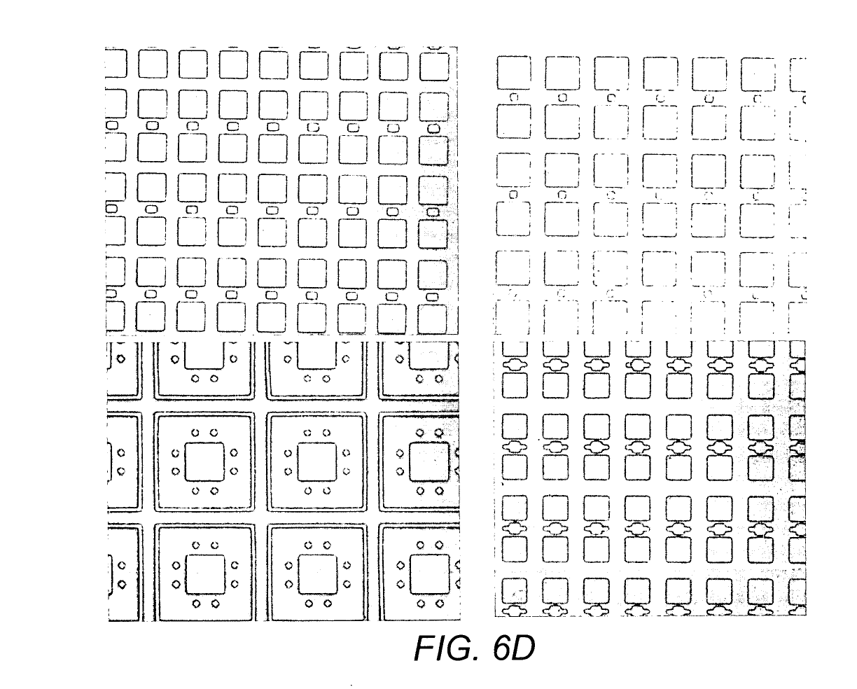

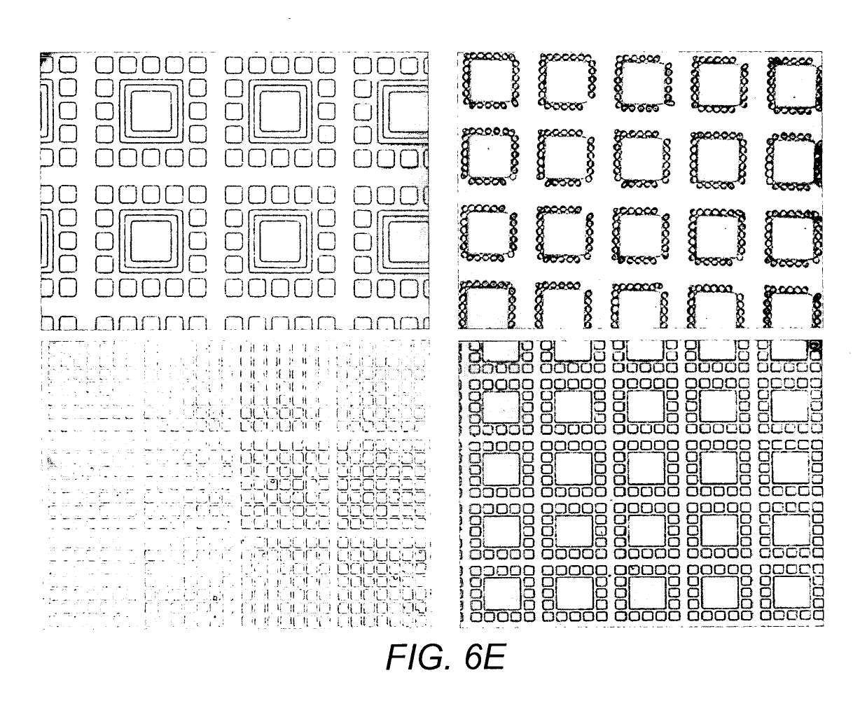

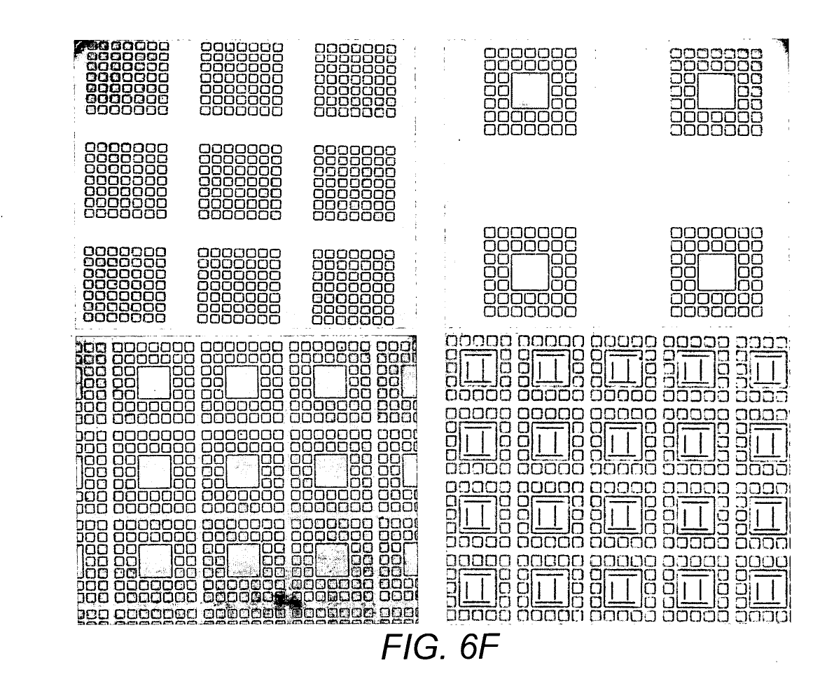

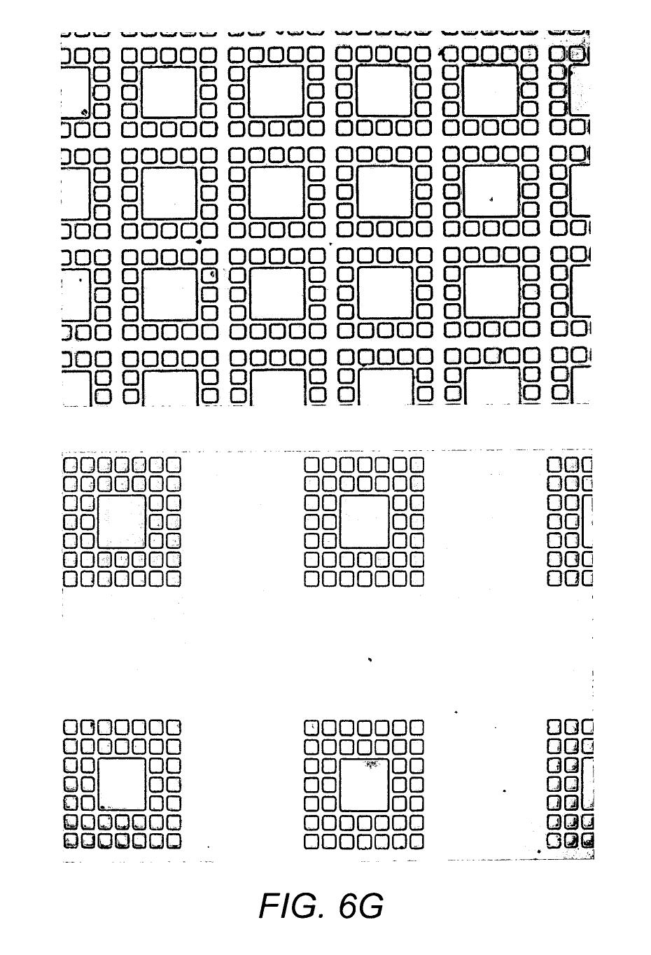

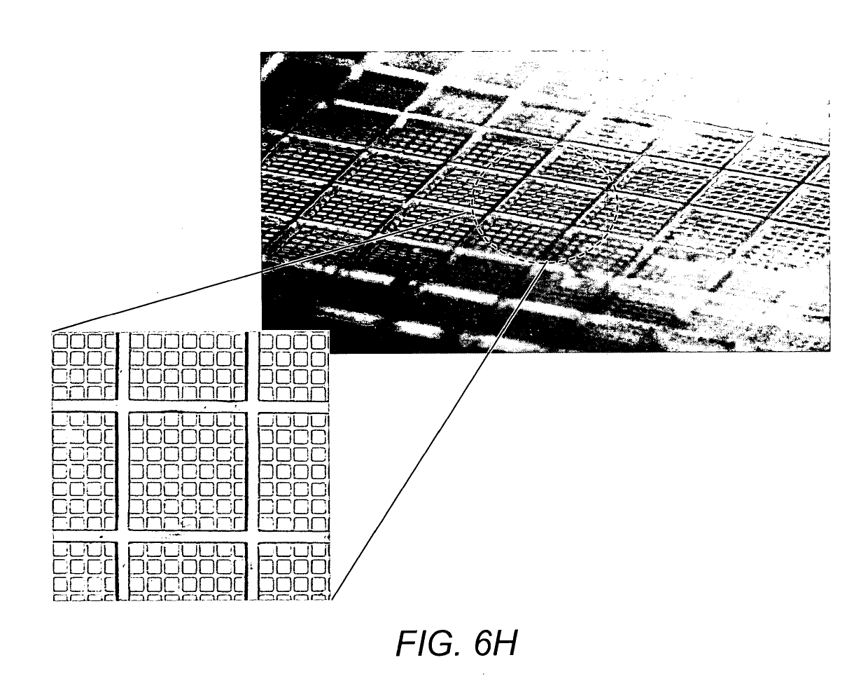

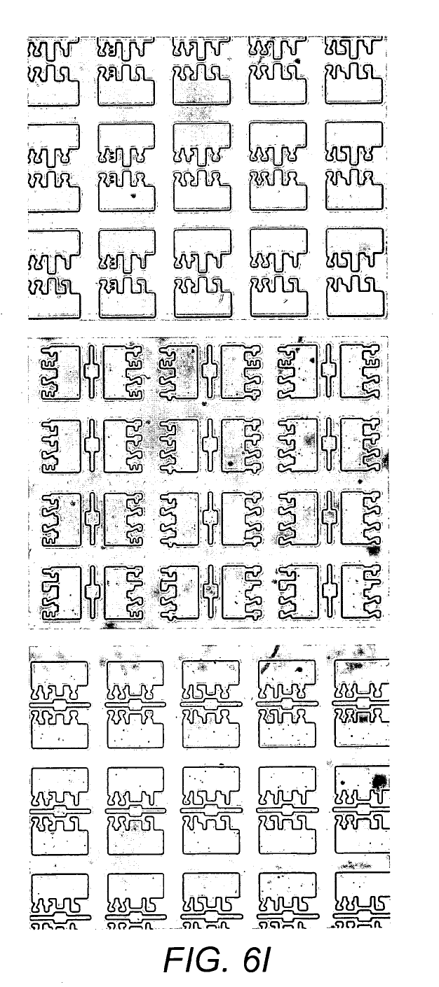

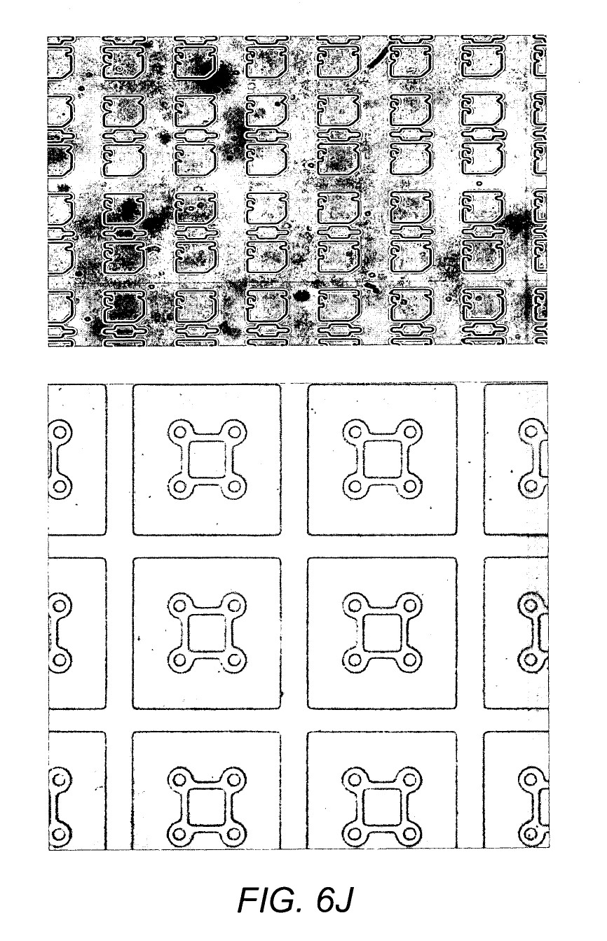

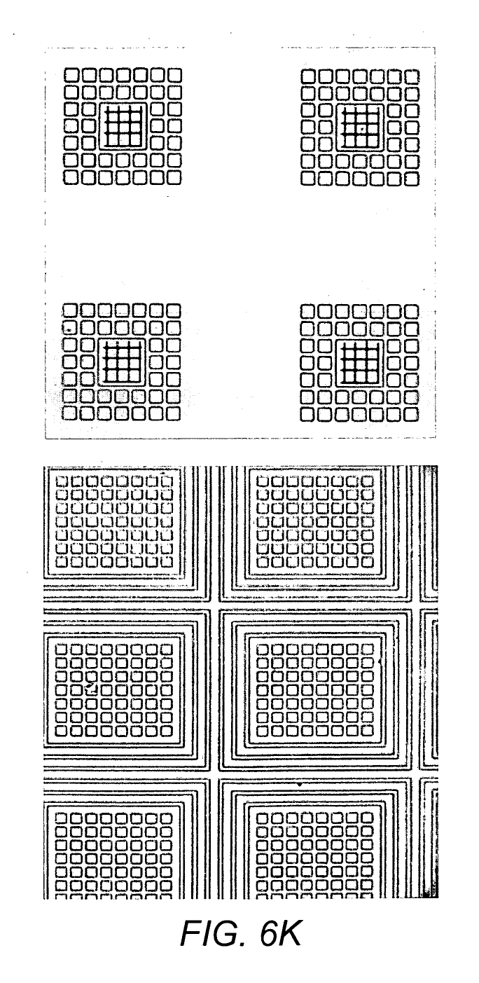

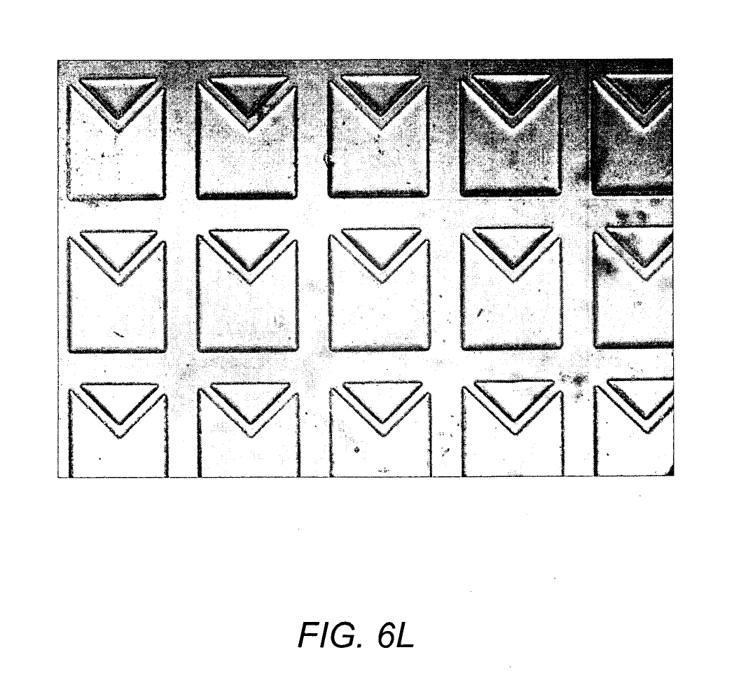

FIGS. 6A-6L show various alternate embodiments of the present invention. In each of the embodiments, there is a clear repeating unit composed of 2 or more pallets (microsctructures) which are not necessarily equal in size. Each repeating unit is the colony growth site. Sampling of the colony then proceeds by sampling one or more of the pallets in that array unit. Some of the embodiments have “cleavage” pallets (i.e. small pallets that are designed and configured to be released and to split the colony apart as the cleavage pallet releases). As an example see FIG. 6A, lower, left array. After the cleavage pallet is released one of the remaining pallet can then be released to sample the colony (now that the colony is split into two fragments on separate pallets).

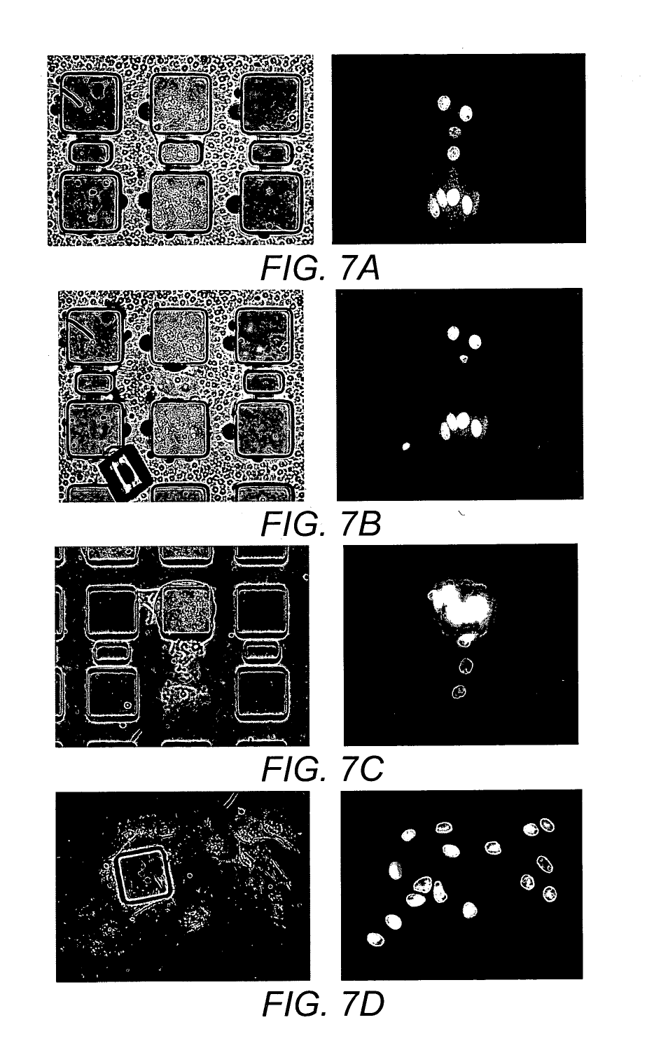

In another example of the invention, a total of 2,000 HeLa GFP cells were plated on a micropallet array containing 6,000 segregated micropallet units. After 72 hr in culture, once the cell colony was observed to appear on the unit, see FIG. 7(a), the cutting micropallet was released by a 10 μJ laser pulse to cut the cell colony. The colony was successfully split into two separate portions. Both portions remained attached to their two respective micropallets and were not detached by the release of the intervening, or cutting, micropallet, see FIG. 7(b). At that point, one of the remaining micropallets containing cells was released and collected in order to sample a portion of the cell colony. The collected micropallet and its attached cells were then cultured in the incubator for another 72 hr and were then were imaged under both transmitted and fluorescent light to verify the viability of the collected cells. The cell colony grown from the sampled portion was clearly observed, see FIG. 7(d). The viability of the remained portion of the cell colonies on the unreleased cell growth micropallets were also tracked after 72 hr in culture. It was found that the unreleased cell colony portions were remained viable, see FIG. 7(c).

The foregoing is illustrative of the present invention, and is not to be construed as limiting thereof. The invention is defined by the following claims, with equivalents of the claims to be included therein.

1. A method for selecting and propagating a cell colony of interest from among a plurality of cell colonies carried on a common substrate in culture, said method comprising the steps of:
(a) selecting a cell colony of interest from among said plurality of cell colonies;
(b) isolating a cell subset from said cell colony of interest;
(c) analyzing said cell subset isolated from said cell colony of interest to confirm the presence or absence of a desired feature therein; and then
(d) propagating said cell colony of interest when said desired feature is present in said cell subset.

2. The method of claim 1, wherein said analysis is a destructive analysis.

3. The method of claim 2, wherein said destructive analysis is selected from the group consisting of PCR, intracellular immunostaining, mass spectrometry, mRNA expression, electron microscopy, electrophoretic analysis, and DNA analysis.

4. The method of claim 1, wherein said desired feature is stable expression of a heterologous nucleic acid.

5. The method of claim 1, wherein said plurality of cell colonies comprises not more than 10 colonies.

6. The method of claim 1, wherein said plurality of cell colonies comprises at least 50 cell colonies.

7. The method of claim 1, wherein said cell colony of interest comprises not more than 100 cells.

8. The method of claim 1, wherein said step of isolating a cell subset is carried out by releasing a portion of cells from said colony of interest, while maintaining the remainder of said colony of interest on said substrate.

9. The method of claim 8, wherein each of said plurality of colonies are grown on a plurality of carriers releasably connected to said common substrate; and
said releasing step is carried out by releasing said carrier from said substrate to thereby release a portion of cells from said colony of interest.

10. The method of claim 9, wherein said carrier is selected from the group consisting of liquid particles, solid particles, and cleavable molecules.

11. The method of claim 8, wherein said releasing a portion is carried out by laser cutting of the colony.

12. A method for isolating a cell subset from a live cell colony grown in culture, comprising the steps of:
(a) providing a micropallet apparatus, said apparatus comprising a:
(i) a substrate;
(ii) a plurality of discrete arrays formed on said substrate, each of said arrays comprising a plurality of pallets releasably connected to said substrate; and
(iii) a cell colony of interest adhered to one of said arrays, said cell colony of interest spanning at least two pallets;
(b) selecting a subset of at least one pallet from said at least two pallets to which said cell colony of interest is adhered;
(c) releasing said selected subset of pallets; and then
(d) collecting at least one cell from said selected subset of pallets to thereby isolate a cell subset from said colony.

13. The method of claim 12, wherein pallets of each of said arrays are separated from one another by gaps.

14. The method of claim 12, wherein said arrays are separated from one another by walls.

15. The method of claim 12, wherein said pallet is connected to said substrate at a release point, and said releasing step is carried out by directing a high energy laser at said release point.

16. The method of claim 12, further comprising:
(e) analyzing said cell subset isolated from said cell colony of interest to confirm the presence or absence of a desired feature therein.

17. The method of claim 16, wherein said analysis is a destructive analysis.

18. The method of claim 17, wherein said destructive analysis is selected from the group consisting of PCR, intracellular immunostaining, mass spectrometry, mRNA expression, electron microscopy, electrophoretic analysis, and DNA analysis.

19. The method of claim 16, wherein said desired feature is stable expression of a heterologous nucleic acid.

20. The method of claim 16 further comprising:
(f) propagating said cell colony of interest when said desired feature is present in said selected cell subset.

21. A micropallet apparatus, comprising:
(a) a substrate;
(b) a plurality of discrete arrays formed on said substrate, each of said arrays comprising a plurality of releasable pallets, and
(c) a plurality of gap forming regions, wherein said gap forming regions surround said pallets and separate said pallets from one another.

22. The micropallet apparatus of claim 21, wherein said pallets are transparent.

23. The micropallet apparatus of claim 21, wherein said pallets are formed from a photoresist resin, a photoactive compound, and a solvent.

24. The micropallet apparatus of claim 23, wherein said pallets are formed from EPON resin 1002F, photoinitiator triarylsulfonium hexafluoroantimonate, and γ-butyrolactone.

25. The micropallet apparatus of claim 21, wherein said pallets have heights in the range of 1 to 400 micrometers.

26. The micropallet apparatus of claim 21, wherein the surface of said pallets is modified to enhance cell culture.

27. The micropallet apparatus of claim 21, wherein said gap forming regions are configured to allow cells to spread over multiple pallets.

28. The micropallet apparatus of claim 21, wherein said gap forming regions are formed from a gas, a liquid, a hydrogel, a solid material or combination thereof.

29. The apparatus of claim 21, further comprising:
(d) a plurality of walls, wherein said walls surround said arrays and separate said arrays from one another.

30. The micropallet apparatus of claim 29, wherein said walls are configured to prevent cell colonies from spreading onto adjacent arrays.

31. The micropallet apparatus of claim 29, wherein said walls are formed from a gas, a hydrogel, a solid material or combination thereof.

32. The micropallet apparatus of claim 29, wherein said walls comprise a cell adhesion resistant material.

33. The micropallet apparatus of claim 32, wherein said material is a PEG hydrogel.

34. The micropallet apparatus of claim 21, further comprising:
a collection plate connected to said micropallet apparatus, said collection plate comprises a plurality of wells, with said plurality of wells are aligned with said plurality of arrays.
